(19) United States
(12) Patent Application Publication
(10) Pub. No.: US 2017/0129827 A1
(43) Pub. Date: May 11, 2017

(54) A PROCESS FOR CONVERTING NATURAL GAS TO HIGHER HYDROCARBON(S)

(71) Applicant: SABIC GLOBAL TECHNOLOGIES B.V., Bergen op Zoom (NL)

(72) Inventor: Christoph Dittrich, Geleen (NL)

(21) Appl. No.: 15/323,587

(22) PCT Filed: Jul. 2, 2015
(86) PCT No.: PCT/EP2015/065070

§ 371 Date: Jan. 3, 2017

(30) Foreign Application Priority Data
Jul. 4, 2014 (EP) 14175735.1

(51) Int. Cl. C07C 2/76; B01J 8/04

(52) U.S. Cl. CPC C07C 2/76; B01J 8/0457; B01J 8/0492; B01J 8/0496; C07C 2529/076; C07C 2527/22; B01J 2208/00044; B01J 2208/00176; B01J 2208/065

(57) ABSTRACT

The present invention relates to a process for converting natural gas to higher hydrocarbon(s) including aromatic hydrocarbon(s) in n reaction zones operated in series, wherein m reaction zones are not participating in the conversion process and only (n−m) reaction zones are operated under reaction conditions sufficient to convert at least a portion of said natural gas to an effluent having said higher hydrocarbon(s). An object of the present invention is to provide a process for converting natural gas to higher hydrocarbon(s) including aromatic hydrocarbon(s) wherein a high reactant, i.e. methane, conversion can be achieved.

The present invention relates to a process for converting natural gas to higher hydrocarbon(s). More in detail, the present invention relates to a process for converting natural gas to higher hydrocarbon(s) including aromatic hydrocarbon(s) in n reaction zones operated in series, wherein m reaction zones are not participating in the conversion process and only (n−m) reaction zones are operated under reaction conditions sufficient to convert at least a portion of said natural gas to an effluent having said higher hydrocarbon(s).

Aromatic hydrocarbons, particularly benzene, toluene, ethylbenzene and xylenes, are important commodity chemicals in the petrochemical industry. Currently, aromatics are most frequently produced from petroleum-based feedstocks by a variety of processes, including catalytic reforming, catalytic cracking, and steam cracking.

A future source of aromatic hydrocarbons can be methane, which is the major constituent of natural gas and biogas. Because of the problems associated with transportation of large volumes of natural gas, a large portion of the natural gas produced along with oil, particularly at remote places, is flared and wasted. Hence the conversion of alkanes contained in natural gas directly to higher hydrocarbons, such as aromatics, is an attractive method of upgrading natural gas. Processes currently proposed for converting methane to liquid hydrocarbons involve initial conversion of the methane to synthesis gas, a blend of H₂ and CO.

U.S. patent application number 2010/305374 relates to a process for converting methane to higher hydrocarbon(s) including aromatic hydrocarbon(s) in two or more reaction zones operated in series, wherein at least a portion of the effluent of the first reaction zone is transferred to a second reaction zone by maintaining the first reaction zone at a lower average temperature than the second reaction zone, wherein the reaction zones are operated under reaction conditions sufficient to convert at least a portion of methane to a first effluent having said higher hydrocarbon(s). According to this U.S. patent application number the counter current flow of the feedstock and the particulate dehydrocyclization catalyst is arranged to produce an inverse temperature profile across dehydrocyclization reaction system, such that, despite the endothermic nature of the dehydrocyclization reaction, the difference between the reaction temperature of the gaseous effluent at the outlet from the dehydrocyclization reaction system and the reaction temperature of the methane-containing feed at the inlet to the dehydrocyclization reaction system is at least +10° C., such as at least +50° C., for example at least +100° C., and even at least +150° C. Such an inverse temperature profile refers to the temperature profile of a series of catalyst zones, wherein the first (as defined by feed inlet) reaction zone is operated at a lower reaction temperature than is or are the subsequent (as defined by process gas outlet) reaction zone(s), namely the inverse of the temperature profile naturally achieved for an endothermic reaction.

Shudo et al. (2009), Int J Hyd En Vol. 34, pp. 4500-4508 disclose the dehydro-aromatization reaction of methane by utilizing biogas and co-produces hydrogen and benzene. The demonstration plant consisted of a pre-treatment unit, a dehydro-aromatization unit and a steam reforming unit.

CN 1401431 relates to a method for the direct synthesis of aromatics from methane, wherein the reactants are continuously fed to a fixed bed reactor.

The present inventors found that steam reformer-type reactors are expensive to build and tedious to maintain, e.g. comparably long shutdown is necessary for replacing the catalyst.

The present inventors also found that circulating fluidized beds or moving beds require engineering of an expensive solids transport system and the catalyst is subjected to mechanical forces (grinding and attrition, dust formation) stronger than in fixed beds. Furthermore, freshly regenerated catalyst contacts already partially converted reactant mixture at high temperature in a countercurrent moving bed with external catalyst reheating. Coking of catalyst will start quickly again. At the fluid inlet/catalyst outlet, deactivated catalyst contacts fresh reactant at low temperature. In other words, the catalyst activity is low at the fluid inlet/catalysts outlet (low temperature+deactivated catalyst) and high at the fluid outlet/catalyst inlet (high temperature+active catalyst). The increasing catalyst activity profile along the reactant flow direction leads to a lower space-time yield, hence a bigger reactor and a lower catalyst utilization rate, compared to a more even activity profile.

An object of the present invention is to provide a process for converting natural gas to higher hydrocarbon(s) including aromatic hydrocarbon(s) wherein a high reactant, i.e. methane, conversion can be achieved.

Another object of the present invention is to provide a process for converting natural gas to higher hydrocarbon(s) including aromatic hydrocarbon(s) wherein coke formation on the catalyst is controlled.

An object of the present invention is to provide a process for converting natural gas to higher hydrocarbon(s) including aromatic hydrocarbon(s) wherein the physical movement of particulate catalyst is avoided.

The present invention thus provides a process for converting natural gas to higher hydrocarbon(s) including aromatic hydrocarbon(s) in n reaction zones operated in series, wherein m reaction zones are not participating in the conversion process and only (n−m) reaction zones are operated under reaction conditions sufficient to convert at least a portion of said natural gas to an effluent having said higher hydrocarbon(s), wherein each reaction zone is initially numbered serially with a designator from 1 to n, the process comprising:

(a) providing a quantity of catalytic material within each reaction zone;

(b) providing to the reaction zone designated as 1 a hydrocarbon feedstock containing natural gas;

(c) heating at least a portion of the effluent of the said reaction zone designated as 1 to the inlet temperature of the reaction zone designated as 2, and more generally, heating at least a portion of the effluent of each reaction zone with a designator equal or smaller than (n−m−1) to the inlet temperature of the reaction zone with a designator larger by one than that of the reaction zone from which said effluent originates;

(d) transferring said at least portion of said effluent of the said reaction zone designated as 1 to said reaction zone designated as 2, and more generally, transferring said at least portion of said reaction zone with a designator equal or smaller than (n−m−1) to the reaction zone with a designator larger by one than that of the reaction zone from which said at least portion of said effluent originates;

(e) maintaining said reaction zone designated as 2 at an average temperature higher than or equal as in reaction zone designated as 1, and more generally, maintaining each reaction zone with a designator equal or smaller than (n−m) at an average temperature higher or equal as in the reaction zone with designator smaller by one than that of said reaction zone,

feeding the effluent from the reaction zone with the designator (n−m) to another process unit,

and regenerating the reaction zones with a designator larger than (n−m), followed by

(f) terminating transferring effluent from the reaction zone with the designator (n−m−1) to the reaction zone with the designator (n−m);

(g) starting regenerating said reaction zone with the designator (n−m) containing deactivated catalytic material;

(h) raising the inlet temperature of each reaction zone with a designator equal or smaller than (n−m−1) to the former inlet temperature of the reaction zones with a designator larger by one than that of said reaction zone, respectively;

(i) changing the value of each designator equal or smaller than (n−1) to a number larger by one than its initial value, and changing the value of the designator with a value of n to 1,

(j) repeating steps (b) to (i).

One or more of the above identified objects can be achieved by the present method. The above described sequence is repeated each time the last reactor through which the reactant/products mixture passes, and hence with the highest average temperature and coke level, needs regeneration. In this way, an apparent cocurrent flow pattern between the reactant/products mixture and the catalyst is established without physical movement of the catalyst with the associated problems of solids handling and catalyst attrition. While passing the (n−m) catalyst beds on stream the reactant/products mixture experiences a saw tooth-like but overall constant or increasing profile of the average temperature. The average bed temperature is defined as arithmetic mean of the reactor inlet and outlet temperature. It should be noted that the above identified parameters “n reaction zones operated in series”, “m reaction zones not participating in the conversion process” and “(n−m) reaction zones operated under reaction conditions” leave room for an embodiment wherein less than (n−m) reaction zones are under regeneration conditions, or an embodiment wherein different types of regeneration methods are applied. The present method is preferably carried out when the n reaction zones are of equal size.

In addition a higher methane conversion can be achieved with an increasing temperature profile because the equilibrium conversion, which represents the driving force for the reaction rate, corresponds to the reactor outlet temperature. It holds for endothermic equilibrium reactions that the equilibrium conversion is higher at higher temperature. A reactor with an increasing temperature profile therefore can provide more driving force for the conversion than an adiabatic reactor with decreasing temperature profile. If the reaction zones are operated adiabatically, the heat of reaction will need to be provided by increasing the temperature of each stream being transferred from each reaction zone with a designator equal to or smaller than (n−m−1) to a reaction zone with a designator larger by one than that of said reaction zone. The total heat of reaction can be evenly distributed over (n−m) reaction zones of equal size with an increasing profile of the average temperature.

The present inventor found that the process of the present invention can also be used for establishing an apparent countercurrent flow pattern between the reactant/product mixture and the catalyst. According to such an apparent cocurrent flow pattern operation the present method comprises the same steps (a) to (e) as discussed before, followed by

(f) terminating transferring effluent from the reaction zone designated as 1 to the reaction zone designated as 2;

(g) starting regenerating the reaction zone designated as 1 containing deactivated catalytic material;

(h) decreasing the inlet temperature of each reaction zone with a designator larger than 1 and equal or smaller than (n−m) to the former inlet temperature of the reaction zones with a designator smaller by one than that of said reaction zone, respectively;

(i) changing the value of each designator equal and larger than 2 to a number smaller by one than its initial value, and changing the value of the designator with value 1 to n,

(j) repeating steps (b) to (i).

In such an apparent countercurrent flow pattern mode, the catalyst in those of the first (n−m) reactions zones with designated by a smaller value is more deactivated and hence less active. It is therefore easier to evenly distribute the total heat of reaction over (n−m) adiabatic reaction zones of equal size, even without an increasing profile of average bed temperatures in reaction zones (n−m).

According to a preferred embodiment of the present invention the reaction zones are adiabatic catalytic fixed bed reaction zones. Adiabatic catalytic fixed bed reaction zones can be axial flow fixed bed reactors or radial flow fixed bed reactors.

The present catalytic material comprises preferably a bifunctional catalyst of molybdenum carbide on zeolite.

The number ratio of reactors on stream (n−m) and in regeneration (m) is determined depending on the ratio of durations of coke build-up during reaction, and purging, cooling down and regeneration. If these durations change, e.g. because a better catalyst is implemented or the operating conditions are changed, the number of reactors on stream (n−m) and in regeneration (m) can be adjusted accordingly without any hardware modification. A new sequence for routing the inter-reactor streams is sufficient. In a preferred embodiment the total number of reaction zones, n, is at least 4, preferably at least 8, wherein said total number of reaction zones not participating in the conversion process is at most 4, preferably at most 2, respectively. However, the present invention is not limited to a specific number of reaction zones or to a specific number of reaction zones not participating in the conversion process.

According to a preferred embodiment step (f) further comprises monitoring the temperature drop along said bed of catalytic material and terminating transferring effluent when said temperature drop comes below a threshold value, or the outlet temperature exceeds a certain threshold value.

According to a preferred embodiment the inlet temperatures of reactor zones 1 to (n−m) are adjusted slightly during the period between two sequence switching events in order to compensate for activity loss of catalyst inside the reaction zones 1 to (n−m) during said period. As a result, the temperature drop along the catalyst beds in reaction zones 1 to (n−m) remains constant or deviates not more than 10% from a constant value.

According to a preferred embodiment step (g) further comprises cooling down (n−m)^{th} reaction zone bed of catalytic material in apparent concurrent flow pattern operation with a purge gas and passing a regeneration gas through the thus cooled bed of catalytic material. It analogously comprises cooling down first reaction zone bed of catalytic material in apparent countercurrent flow pattern operation.

Coke formation increases with higher temperatures and limits the reaction rate. It is secured with the increasing temperature profile and simulated cocurrent flow that the catalyst is hardly deactivated by coke at low temperature, because it was just regenerated, and most deactivated at high temperature where reaction rates are high anyway. The hottest and most deactivated catalyst is the first to be regenerated in the next sequence. Hence, the catalyst activity decreases together with increasing temperature in flow direction of the reactant/product mixture.

The catalyst is regenerated by coke removal via combustion or hydrogenolysis. If the catalyst is not heat sensitive, or does not form volatile components with oxygen or steam, (enriched) air, or mixtures of steam and (enriched) air can be used to convert coke into carbon oxides. If the catalyst forms volatile components with oxygen, hydrogen-rich gas can be used to convert coke into methane or other hydrocarbons by hydrogenolysis.

Catalyst regeneration by both, combustion or hydrogenolysis, is exothermic and heats up the adiabatic fixed beds. It therefore can be necessary to cool down the catalyst bed prior to regeneration such that it reaches the desired temperature level after regeneration and prior to the next reaction sequence. The reactor to be cooled down can serve as a feed preheater to the first reactor in operation simply by switching off the preheater of the reactor to be cooled down when the end of a sequence approaches.

The regeneration gas is preferably chosen from the group of steam, air and hydrogen, or suitable mixtures thereof.

In a preferred embodiment the regeneration gas comprises at least two different components, wherein the different components are dosed together or the different components are dosed in a sequence.

Catalyst regeneration by coke hydrogenolysis with hydrogen-rich regeneration gas followed by coke combustion with oxygen-containing regeneration gas can be necessary if hydrogenolysis alone cannot recover the catalyst activity anymore. It is often beneficial to regenerate the catalyst by burning off coke at not too high temperature in order to avoid that the large combustion exotherm irreversibly damages the catalyst. Some active catalyst components form volatile oxides during regeneration with oxygen-containing regeneration gas. It is often beneficial to regenerate the catalyst at a temperature low enough to avoid the loss of oxides of the active components by evaporation or sublimation before said oxides are transferred into an active and thermally more stable state after regeneration with oxygen-containing gas.

In a preferred embodiment regeneration by coke combustion with oxygen-containing regeneration gas is carried out at a temperature lower than the minimum temperature at which the reaction in reaction zone 1 to (n−m) or catalyst regeneration by coke hydrogenolysis with hydrogen-rich gas in the remaining reaction zones takes place. In practice, each reaction zone is regenerated by coke combustion with oxygen-containing gas occasionally. For this, the reaction zone to be regenerated with oxygen-rich gas needs to be cooled down to a lower temperature before burning off coke, regenerated with oxygen-rich gas and reheated to a higher temperature. Additionally, oxides of active catalyst components need to be transferred into an active and thermally more stable state, for example by carburization with natural gas, after or during reheating. Cooling down the catalyst bed of said reaction zone, regenerating, reheating and carburization might take longer than the period between two sequence switching events.

In a preferred embodiment each reaction zone is taken out of the sequence of reaction and regeneration at temperatures equal or higher as in reaction for the period of a number of sequence switching events. Said reaction zone is cooled down, regenerated with a different regeneration gas at a lower temperature, reheated and, if necessary, carburized, during said period. After said reaction zone has reached a temperature high enough for the reaction again, it participates in the sequence of reaction and regeneration at temperatures equal or higher as in reaction again, and another reaction zone is taken out of the sequence of reaction and regeneration at temperatures equal or higher as in reaction.

The regeneration sequence of the (n)^{th} reaction zone may further comprise a recarburization step of the catalyst with feed, if necessary.

The present invention will now be discussed by way of an example.

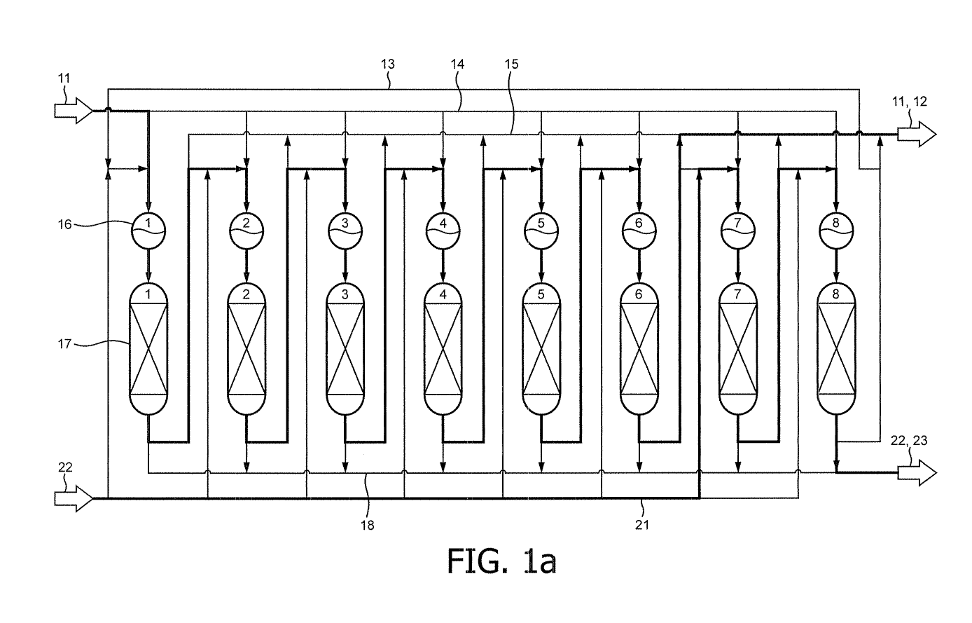

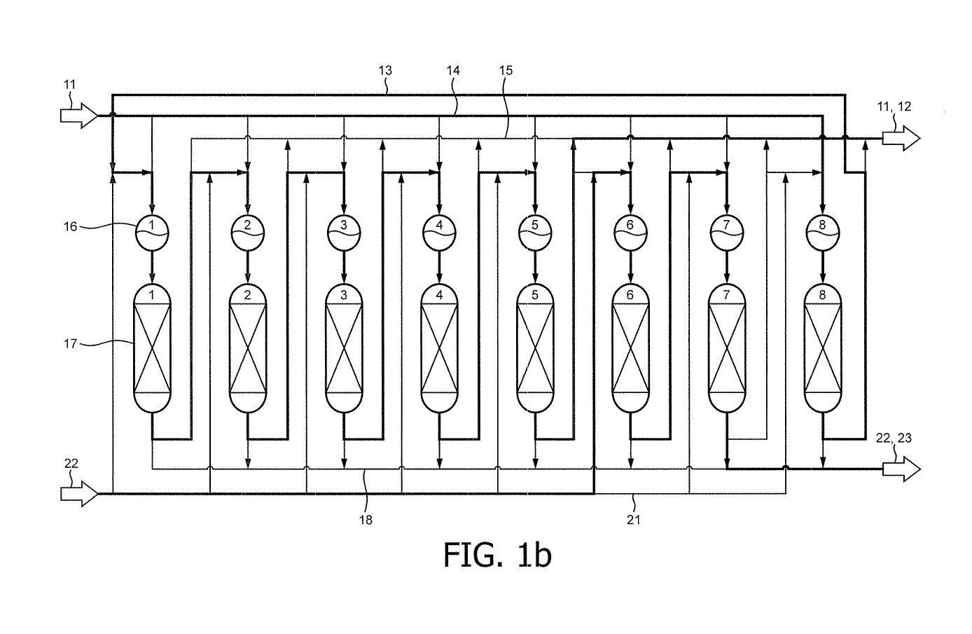

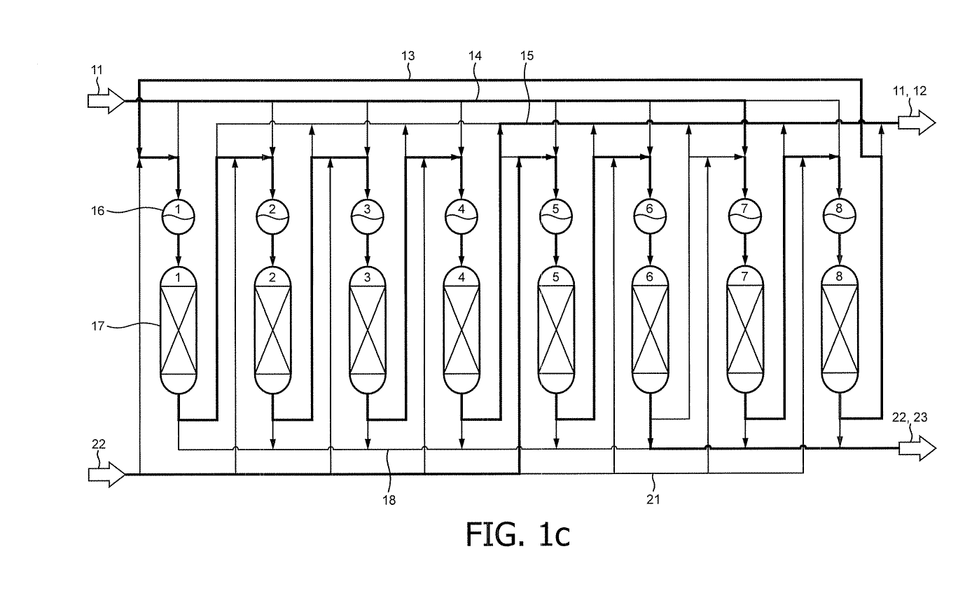

FIG. 1a shows an embodiment of a process for converting natural gas to higher hydrocarbon(s) including aromatic hydrocarbon(s).

FIG. 1b shows another phase of the same process for converting natural gas to higher hydrocarbon(s) including aromatic hydrocarbon(s).

FIG. 1c shows another phase of the same process for converting natural gas to higher hydrocarbon(s) including aromatic hydrocarbon(s).

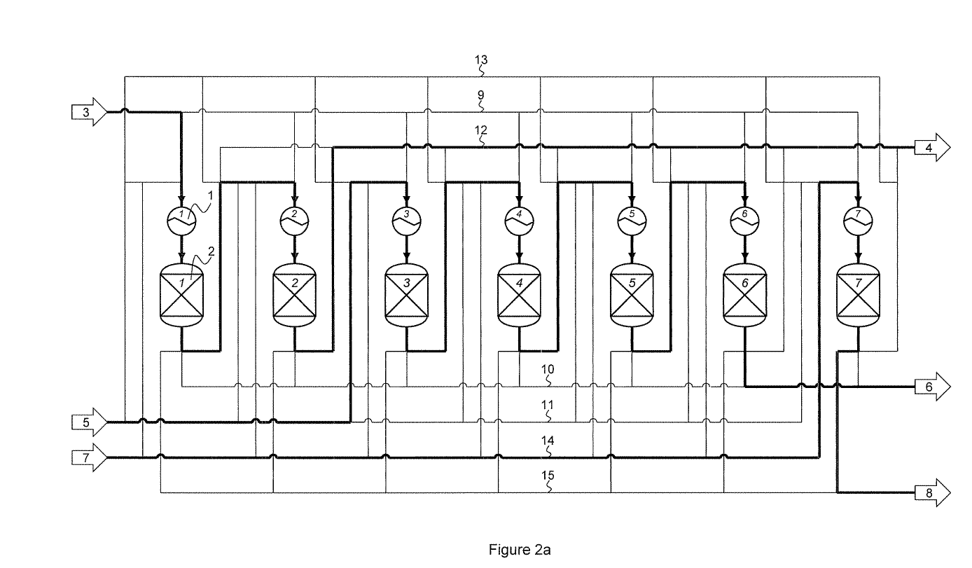

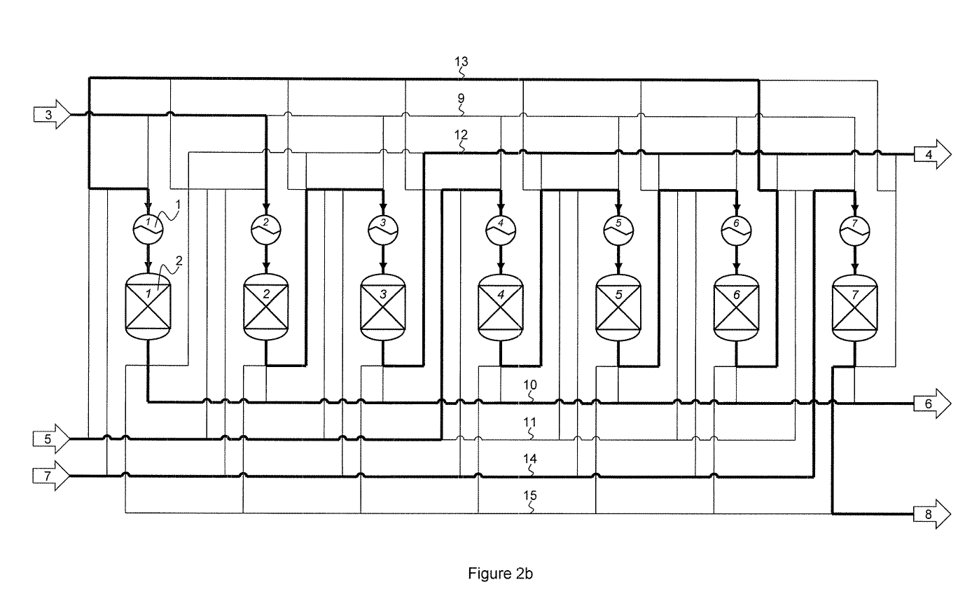

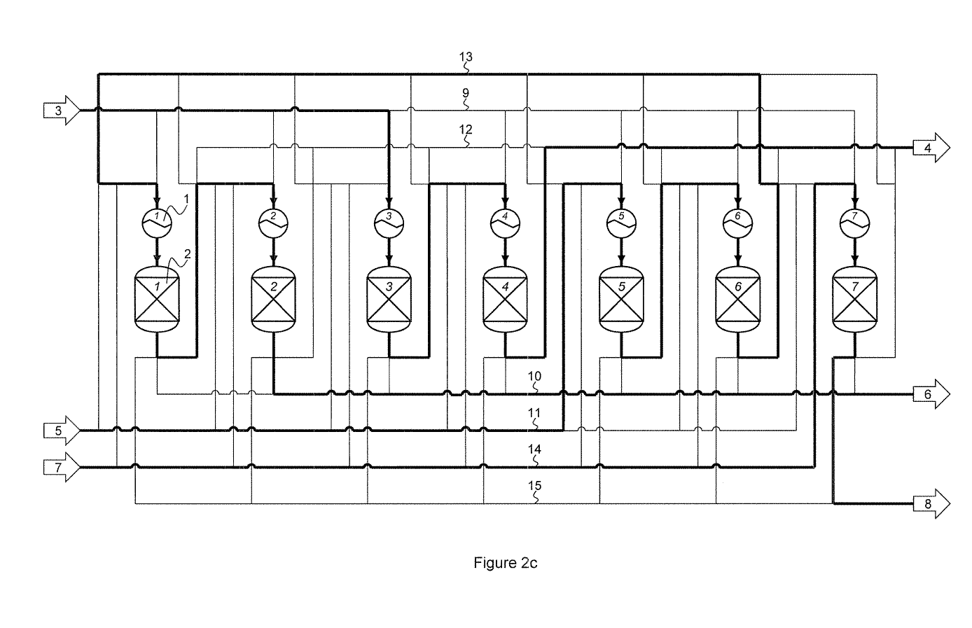

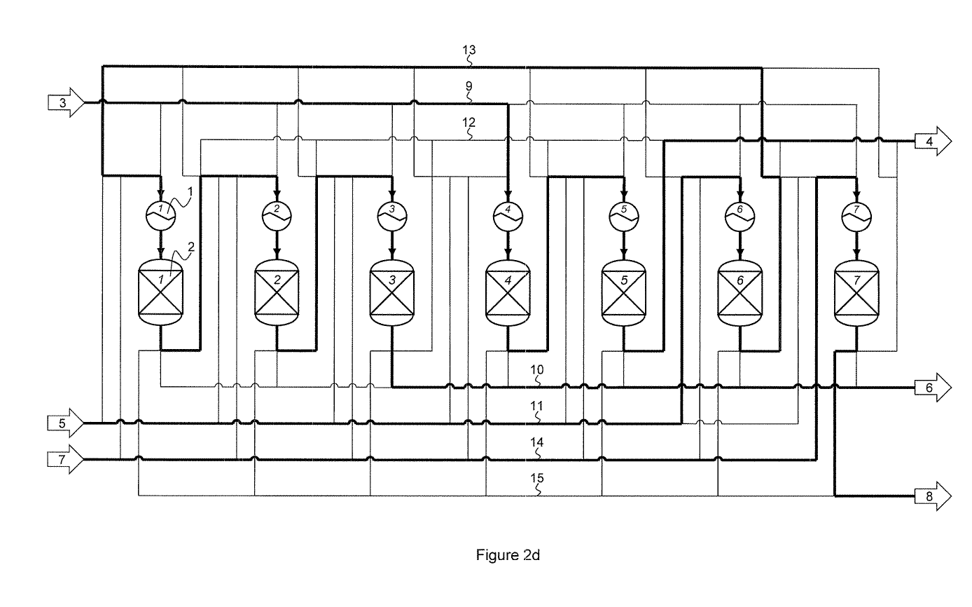

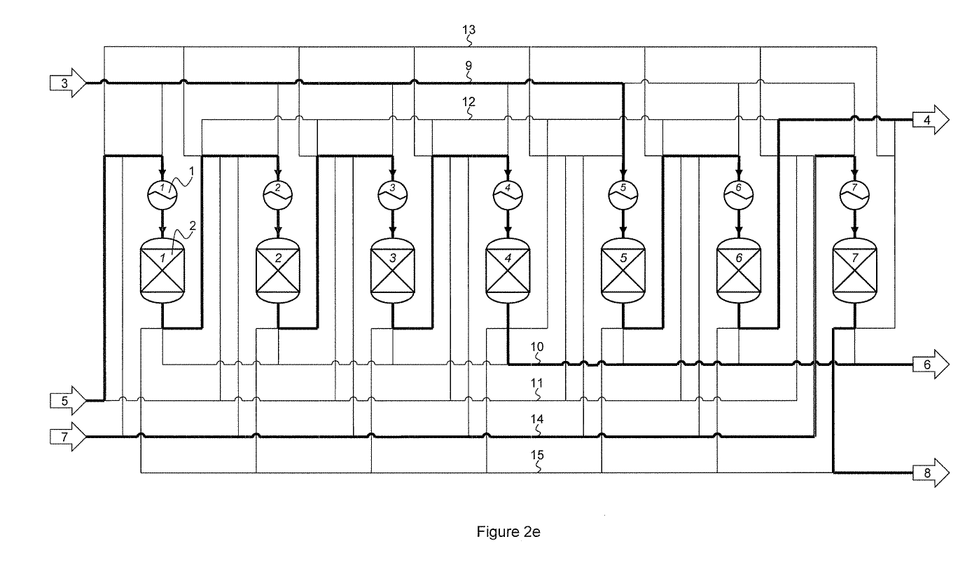

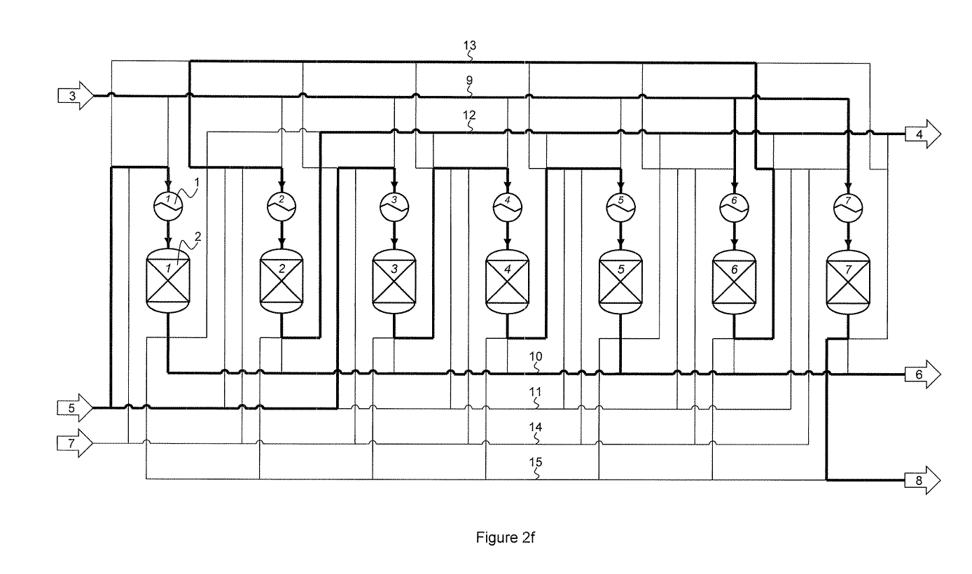

FIGS. 2a-2g show different sequences of another embodiment of a process for converting natural gas to higher hydrocarbon(s) including aromatic hydrocarbon(s).

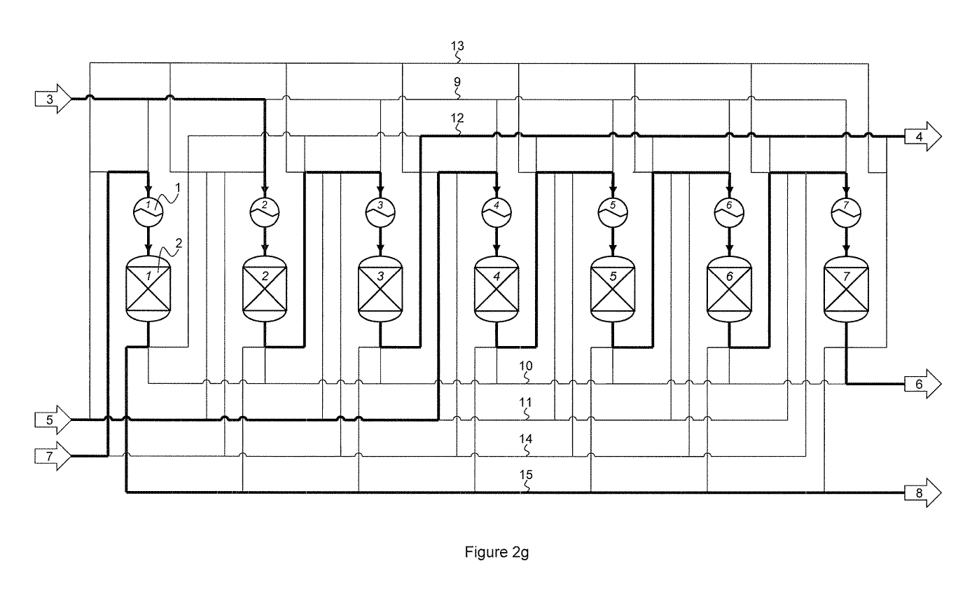

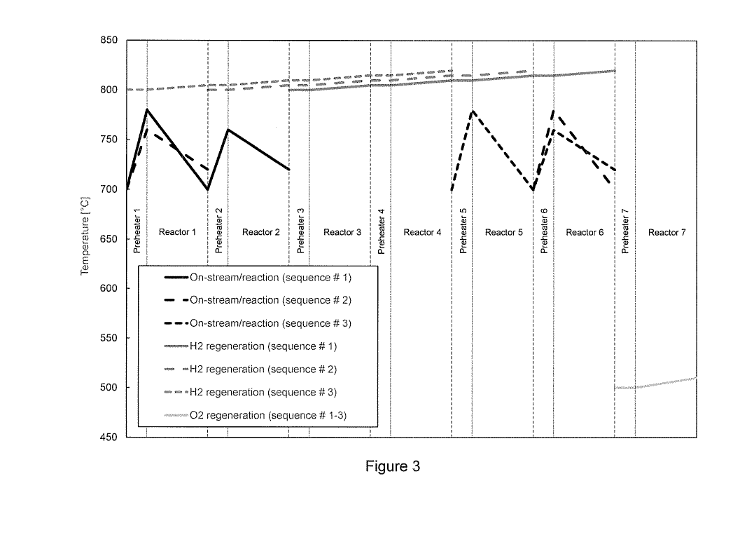

FIG. 3 illustrates the temperature profile during the first three sequences shown in FIGS. 2a-2c.

Natural gas is fed to the first of a series of n adiabatic catalytic fixed bed reactors. The feed is brought to a preset temperature in the preheater of the first reactor. Each reactor contains particulate catalyst, e.g. a bifunctional catalyst of molybdenum carbide on zeolite, which converts methane and lower hydrocarbons into benzene and other higher hydrocarbons. m reactors are being regenerated in order to remove coke from the catalyst while n−m reactors are on stream, which means convert natural gas into aromatics.

The mixture of reactant and nascent products passes through the first adiabatic reactor where it cools down due to the endothermic nature of the reaction. Hence, a decreasing temperature profile along the flow direction is established inside the catalytic fixed bed of the first reactor. After leaving the first reactor, the effluent is heated up again and enters the second reactor. More natural gas is converted into aromatics in the second reactor where the reactant/product mixtures cool down and a declining temperature profile is again established. The outlet temperature of the second reactor is higher than the outlet temperature of the first reactor. The sequence of reheating the reactant/product mixture, the conversion of the reheated mixture inside each reactor accompanied by temperature decrease, and the converted mixture leaving each reactor at a higher temperature than the outlet temperature of the previous reactor is repeated according to the total number (n−m) of reactors on stream.

The detailed discussion of the FIGS. 1a-1b-1c relates to the specific embodiment of the present invention in which the overall temperature profile increases.

### EXAMPLE 1

A first example is given in FIG. 1a with n=8 reactors of which m=2 reactors are regenerated. The effluent of the last (n−m)^{th} reactor on stream is quenched and fed to the product separation section of the process. As a result of the increasing profile of the inlet temperatures of each of the (n−m) reactors on stream the catalytic fixed bed of the (n−m)^{th} reactor has the highest average temperature. This implies that the rate of coke formation, which is an undesired side reaction, is highest and therefore the catalyst deactivates most quickly in the (n−m)^{th} reactor.

When a preset minimum conversion is reached indicated by a minimum temperature drop along the catalytic bed of the (n−m)^{th} reactor as result of catalyst deactivation, the (n−m)^{th} reactor will be taken off stream and regenerated. For this, the catalytic bed is first cooled down by a purge gas, e.g. cold methane without preheating, and then the catalyst is regenerated by converting coke with a regeneration gas. At the same time, all inlet temperature set points of the first to (n−m−1)^{th} reactor are raised to the values of the second to (n−m)^{th} reactor before the (n−m)^{th} reactor was taken off stream for regeneration. The feed stream is not fed to the first reactor anymore but to the n^{th} reactor which has been regenerated until now. The inlet temperature set point of the n^{th} reactor is now the same as for the first reactor before the (n−m)^{th} reactor was taken off stream for regeneration. According to FIG. 1B the effluent of the n^{th} reactor is now routed to the first reactor (n=8 and m=2).

The (n−m−1)^{th} reactor has the highest average temperature now and will be the next one to be regenerated. Once the temperature drop inside the (n−m−1)^{th} reaches a preset minimum value the same switching sequence is triggered like described above. After the new switching event, as shown in FIG. 1c, the feed will be routed to the (n−1)^{th} reactor, the average temperature level in the (n−m−2)^{th} reactor will be the highest and the effluent of the (n−m−2)^{th} reactor is fed to the product separation section (n=8 and n=2).

The process as discussed above has been disclosed in Table .

TABLE 1
Schematic overview of status of reaction zones
in the process according to the invention

| | | Reaction zone (number) | | | | | | | |
|---|---|---|---|---|---|---|---|---|---|
| | | 1 | 2 | 3 | 4 | 5 | 6 | 7 | 8 |
| Cycle | 1 | R | R | R | R | R | R | X | X |
| | 2 | R | R | R | R | R | X | X | R |
| | 3 | R | R | R | R | X | X | R | R |
| | 4 | R | R | R | X | X | R | R | R |
| | 5 | R | R | X | X | R | R | R | R |
| | 6 | R | X | X | R | R | R | R | R |
| | 7 | X | X | R | R | R | R | R | R |
| | 8 | X | R | R | R | R | R | R | X |
| | 9 | R | R | R | R | R | R | X | X |

From Table 1 one can deduce that in the first cycle only reaction zones 1-6 are participating in the conversion process whereas reaction zones 7-8 are not participating in the conversion process. The sequence in the first cycle is thus 1-2-3-4-5-6 (on stream) and 7-8 (off stream). In the second cycle reaction zone 8 is taken off stream and the “first” reaction zone will now be reaction zone 8. The effluent from reaction zone 8 is fed to the inlet of second reaction zone, now being reaction zone 1. The sequence in the second cycle is thus 8-1-2-3-4-5 (on stream) and 6-7 (off stream), wherein the highest temperature is in reaction zone 5. The sequence in the third cycle is thus 7-8-1-2-3-4 (on stream) and 5-6 (off stream), wherein the highest temperature is in reaction zone 6. This table 1 shows a number of nine cycles wherein the situation of cycle 1 is similar to cycle 9. As mentioned before, the present invention is not restricted to any specific number of reaction zones.

In the FIGS. 1a-1c the reference numbers used refer to the following:

11=Natural gas

12=Products

13=Effluent from reactor 8=Feed to reactor 1 (ring main)

14=Feed header

15=Product header

16=Preheater

17=Reactor

18=Regeneration effluent header

21=Regeneration gas header

22=Regeneration gas

23=Decoking products

### EXAMPLE 2

A second example is given in FIG. 2a-2g with n=7 reactors of which m=4 reactors are regenerated with hydrogen-containing gas and k=1 is regenerated with oxygen-containing gas.

In the FIGS. 2a-2g the reference numbers used refer to the following (please note that these numbers do not refer to the numbers used in Example 1):

1 Preheater 1 to n (7)

2 Reaction zone 1 to n

3 Natural gas feed

4 Product mixture

5 Hydrogen-rich regeneration gas feed

6 Hydrogen-rich regeneration gas effluent

7 Oxygen-containing regeneration gas feed

8 Oxygen-containing regeneration gas effluent

9 Natural gas distribution line

10 Product collection line

11 Hydrogen-rich regeneration gas distribution line

12 Hydrogen-rich regeneration gas collection line

13 Ring main

14 Oxygen-containing regeneration gas distribution line

15 Oxygen-containing regeneration gas collection line

The effluent of the last (second) reactor on stream is quenched and fed to the product separation section of the process. The catalyst beds of each of the two reactors on stream have the same average temperature. The catalyst in the first reactor has accumulated more coke and is more deactivated than that in the second reactor because it has been on stream for longer.

When a preset minimum conversion is reached indicated by a minimum temperature drop along the catalytic bed of the first reactor as result of catalyst deactivation, the second reactor will be taken off stream and regenerated. For this, the catalyst is regenerated by converting coke with a hydrogen-rich regeneration gas. At the same time, the inlet temperature set point of the second reactor is adjusted to the value of the first reactor before it was taken off stream for regeneration. The feed stream is not fed to the first reactor anymore but to the second reactor. The inlet temperature set point of the second reactor is now the same as for the first reactor before it was taken off stream for regeneration. According to FIG. 2b the effluent of the second reactor is now routed to the third reactor.

The catalyst bed of the second reactor has been on stream for the longest time now and will be the next one to be regenerated. Once the temperature drop inside the second reactor reaches a preset minimum value the same switching sequence is triggered like described above. After the new switching event, as shown in FIG. 2c, the feed will be routed to the third reactor, the catalyst bed in the third reactor will have been on stream for the longest time and the effluent of the fourth reactor is fed to the product separation section.

FIGS. 2d, 2e and 2f show how the feed and effluent streams are routed during the next three sequences. After the sixth (n−k)th switching event, the first sequence (FIG. 2a) is repeated.

The seventh (n)th reactor is regenerated with oxygen-containing regeneration gas, e.g. diluted air, at a lower temperature and does not participate in each switching sequence. When regeneration by oxygen is completed, the catalyst bed of the seventh reactor is carburized and reheated with a hydrocarbon-rich gas, e.g. natural gas feed, and put back into the series of reactors participating in the switching sequence, i.e. on stream or regenerated by hydrogen-rich gas. At the same time, the first reactor is put out of the series of reactors participating in the switching sequence, cooled down, e.g. with cold natural gas feed, and regenerated with oxygen-containing gas, e.g. diluted air. FIG. 2f shows that feed of oxygen-containing regeneration gas to the seventh reactor has stopped and feed of natural gas to the seventh reactor for catalyst carburization has started. FIG. 2g shows how feed and effluent streams are routed after putting the seventh reactor out of and the first reactor into the switching sequence after the previous cycle of six (n−k) switching sequences has finished.

Table 2 gives an overview of a complete cycle of switching events (sequence #1 to 6), and the start of a second cycle with the second to seventh reactor participating in the switching sequences (sequences #7 and later). After the first complete cycle (sequences #1 to 6), the seventh reactor is put into and the first reactor out of the series of reactors participating in the switching sequences (sequence #7).

TABLE 2
Schematic overview of status of reaction zones in the process
according to the invention according to another embodiment

| | Reactor | | | | | | | |
|---|---|---|---|---|---|---|---|---|
| Sequence | # | | | | | | | |
| # | 1 | 2 | 3 | 4 | 5 | 6 | 7 | FIG. |
| 1 | Reaction | Reaction | H2 | H2 | H2 | H2 | O2 | 2a |
| | | | Regen | Regen | Regen | Regen | Regen | |
| 2 | H2 | Reaction | Reaction | H2 | H2 | H2 | O2 | 2b |
| | Regen | | | Regen | Regen | Regen | Regen | |
| 3 | H2 | H2 | Reaction | Reaction | H2 | H2 | O2 | 2c |
| | Regen | Regen | | | Regen | Regen | Regen | |
| 4 | H2 | H2 | H2 | Reaction | Reaction | H2 | O2 | 2d |
| | Regen | Regen | Regen | | | Regen | Regen | |
| 5 | H2 | H2 | H2 | H2 | Reaction | Reaction | O2 | 2e |
| | Regen | Regen | Regen | Regen | | | Regen | |
| 6 | H2 | Reaction | H2 | H2 | H2 | Reaction | Carbu- | 2f |
| | Regen | | Regen | Regen | Regen | | rization | |
| 7 | O2 | Reaction | Reaction | H2 | H2 | H2 | H2 | 2g |
| | Regen | | | Regen | Regen | Regen | Regen | |
| 8 | O2 | H2 | Reaction | Reaction | H2 | H2 | H2 | — |
| | Regen | Regen | | | Regen | Regen | Regen | |
| 9 | O2 | H2 | H2 | Reaction | Reaction | H2 | H2 | — |
| | Regen | Regen | Regen | | | Regen | Regen | |
| . . . | . . . | . . . | . . . | . . . | . . . | . . . | . . . | — |

In Table 2the term “Regen” means regeneration, the term “H2” means hydrogen, the term “O2” means oxygen.

FIG. 3 illustrates the temperature profile during the first three sequences shown in FIGS. 2a -2c, and in table 2.

1. A process for converting natural gas to higher hydrocarbon(s) including aromatic hydrocarbon(s) in n reaction zones operated in series, wherein m reaction zones are not participating in the conversion process and only (n−m) reaction zones are operated under reaction conditions sufficient to convert at least a portion of said natural gas to an effluent having said higher hydrocarbon(s), wherein each reaction zone is initially numbered serially with a designator from 1 to n, the process comprising:
(a) providing a quantity of catalytic material within each reaction zone;
(b) providing to the reaction zone designated as 1 a hydrocarbon feedstock containing natural gas;
(c) heating at least a portion of the effluent of the said reaction zone designated as 1 to the inlet temperature of the reaction zone designated as 2, and more generally, heating at least a portion of the effluent of each reaction zone with a designator equal or smaller than (n−m−1) to the inlet temperature of the reaction zone with a designator larger by one than that of the reaction zone from which said effluent originates;
(d) transferring said at least portion of said effluent of the said reaction zone designated as 1 to said reaction zone designated as 2, and more generally, transferring said at least portion of said reaction zone with a designator equal or smaller than (n−m−1) to the reaction zone with a designator larger by one than that of the reaction zone from which said at least portion of said effluent originates;
(e) maintaining said reaction zone designated as 2 at an average temperature higher than or equal as in reaction zone designated as 1, and more generally, maintaining each reaction zone with a designator equal or smaller than (n−m) at an average temperature higher or equal as in the reaction zone with designator smaller by one than that of said reaction zone,
feeding the effluent from the reaction zone with the designator (n−m) to another process unit,
and regenerating the reaction zones with a designator larger than (n−m), followed by
(f) terminating transferring effluent from the reaction zone with the designator (n−m−1) to the reaction zone with the designator (n−m);
(g) starting regenerating said reaction zone with the designator (n−m) containing deactivated catalytic material;
(h) raising the inlet temperature of each reaction zone with a designator equal or smaller than (n−m−1) to the former inlet temperature of the reaction zones with a designator larger by one than that of said reaction zone, respectively;
(i) changing the value of each designator equal or smaller than (n−1) to a number larger by one than its initial value, and changing the value of the designator with a value of n to 1,
(j) repeating steps (b) to (i).

2. A process for converting natural gas to higher hydrocarbon(s) including aromatic hydrocarbon(s) in n reaction zones operated in series, wherein m reaction zones are not participating in the conversion process and only (n−m) reaction zones are operated under reaction conditions sufficient to convert at least a portion of said natural gas to an effluent having said higher hydrocarbon(s), wherein each reaction zone is initially numbered serially with a designator from 1 to n, the process comprising:
(a) providing a quantity of catalytic material within each reaction zone;
(b) providing to the reaction zone designated as 1 a hydrocarbon feedstock containing natural gas;
(c) heating at least a portion of the effluent of the said reaction zone designated as 1 to the inlet temperature of the reaction zone designated as 2, and more generally, heating at least a portion of the effluent of each reaction zone with a designator equal or smaller than (n−m−1) to the inlet temperature of the reaction zone with a designator larger by one than that of the reaction zone from which said effluent originates;
(d) transferring said at least portion of said effluent of the said reaction zone designated as 1 to said reaction zone designated as 2, and more generally, transferring said at least portion of said reaction zone with a designator equal or smaller than (n−m−1) to the reaction zone with a designator larger by one than that of the reaction zone from which said at least portion of said effluent originates;
(e) maintaining said reaction zone designated as 2 at an average temperature higher than or equal as in reaction zone designated as 1, and more generally, maintaining each reaction zone with a designator equal or smaller than (n−m) at an average temperature higher or equal as in the reaction zone with designator smaller by one than that of said reaction zone,
feeding the effluent from the reaction zone with the designator (n−m) to another process unit,
and regenerating the reaction zones with a designator larger than (n−m), followed by
(f) terminating transferring effluent from the reaction zone designated as 1 to the reaction zone designated as 2;
(g) starting regenerating the reaction zone designated as 1 containing deactivated catalytic material;
(h) decreasing the inlet temperature of each reaction zone with a designator larger than 1 and equal or smaller than (n−m) to the former inlet temperature of the reaction zones with a designator smaller by one than that of said reaction zone, respectively;
(i) changing the value of each designator equal and larger than 2 to a number smaller by one than its initial value, and changing the value of the designator with value 1 to n,
(j) repeating steps (b) to (i).

3. The process according to claim 1, wherein said reaction zones are adiabatic catalytic fixed bed reaction zones.

4. The process according to claim 3, wherein said adiabatic catalytic fixed bed reaction zones are adiabatic radial flow fixed bed reactors.

5. The process according to claim 1, wherein said catalytic material comprises a bifunctional catalyst of molybdenum carbide on zeolite.

6. The process according to claim 1, wherein said total number of reaction zones, n, is at least 4, wherein said total number of reaction zones not participating in the conversion process is at most 6, respectively.

7. The process according to claim 1, wherein step (f) further comprises monitoring the temperature drop along said bed of catalytic material and terminating transferring effluent when said temperature drop comes below a threshold value.

8. The process according to claim 1, wherein the inlet temperature of each reaction zone not in regeneration is continuously adjusted in small steps such that the temperature drop along the catalyst bed in said reaction zone deviates not more than 10% from a constant value.

9. The process according to claim 1, wherein a (n+1)th reaction zone exists which is operated at a lower temperature.

10. The process according to claim 1, wherein in said step (g) of regenerating a regeneration gas is chosen from the group of steam, air and hydrogen, or suitable mixtures thereof.

11. The process according to claim 10, wherein said regeneration gas comprises at least two different components, said different components are dosed together.

12. The process according to claim 10, wherein said regeneration gas comprises at least two different components, said different components are dosed in a sequence.

13. The process according to claim 1, wherein the exothermic heat originating from regenerating said bed of catalytic material is used for preheating of fresh feed to the first reactor on stream.

14. The process according to any one of claim 1, wherein regeneration by coke combustion with an oxygen-containing regeneration gas is carried out at a temperature lower than the minimum temperature at which the reaction in reaction zone 1 to (n−m) or catalyst regeneration by coke hydrogenolysis with hydrogen-rich gas in the remaining reaction zones takes place.

15. The process according to claim 6, wherein said total number of reaction zones is at least 7.

16. The process according to claim 6, wherein said total number of reaction zones not participating in the conversion process is at most 4.
